(19) United States
(12) Patent Application Publication
(10) Pub. No.: US 2023/0085402 A1
(43) Pub. Date: Mar. 16, 2023

(54) BIOMARKERS

(71) Applicant: Oxford University Innovation Limited, Oxford (Oxfordshire) (GB)

(72) Inventors: Jeremy TOMLINSON, Oxford (Headington) (GB); Ahmad MOOLLA, Oxford (Headington) (GB)

(21) Appl. No.: 17/796,288

(22) PCT Filed: Jan. 29, 2021
(86) PCT No.: PCT/GB2021/050217

§ 371 Date: Jul. 29, 2022

(30) Foreign Application Priority Data
Jan. 31, 2020 (GB) 2001361.1
Apr. 14, 2020 (GB) 2005435.9

(51) Int. Cl. G01N 33/74

(52) U.S. Cl. CPC G01N 33/743; G01N 2800/52; G01N 2800/085

(57) ABSTRACT

A method of diagnosing non-alcoholic fatty liver disease (NAFLD) in a subject, and/or determining the stage of NAFLD in a subject diagnosed with NAFLD; or a method of identifying a subject having an increased risk of developing liver cancer; or a method of treating a subject with NAFLD having advanced fibrosis or cirrhosis; wherein the method comprises determining the level of at least one steroid hormone or metabolite thereof in a urine sample provided by the subject.

### TECHNICAL FIELD

The present invention relates to novel urinary biomarkers for use in assessing the stage of non-alcoholic fatty liver disease in a subject: or for identifying a subject having an increased risk of developing liver cancer; or a method of treating a subject with NAFLD having advanced fibrosis or cirrhosis

### INTRODUCTION

Ectopic fat deposition in the liver, known as non-alcoholic fatty liver disease (NAFLD), affects up to 30% of the worldwide population, and up to 70% of patients with type 2 diabetes mellitus (T2D), rising to more than 90% of patients undergoing weight loss surgery. By 2025, it is estimated that NAFLD will be the leading cause of liver failure and the leading indication for liver transplantation. Despite the impact upon the liver, the vast majority of the morbidity and mortality in patients with NAFLD is driven through adverse cardiovascular outcomes.

NAFLD is a spectrum of diseases, ranging from simple steatosis, through to inflammation (steatohepatitis/non-alcoholic steatohepatitis) and subsequently fibrosis, potentially leading to the development of cirrhosis and the associated risk of hepatocellular carcinoma (HCC). There is now clear evidence that morbidity and mortality (both cardiovascular and liver) are increased with progressive worsening of fibrosis and that the drivers to progressive disease include the development of T2D and weight gain.

Despite the adverse clinical outcome, NAFLD is often asymptomatic until its late stages when either liver failure or cardiovascular complications may become apparent. Accurate and early staging is therefore important to determine patient risk of complications and to guide the most appropriate management strategy. The current gold standard for staging liver fibrosis in patients with NAFLD remains a liver biopsy, which is invasive, associated with morbidity, resource intensive and samples only a very small fraction of the liver and therefore may be prone to error.

Routine liver biochemistry is unhelpful in staging NAFLD; 50% of patients with advanced fibrosis or cirrhosis may have entirely normal liver chemistry. Faced with this challenge, several non-invasive tools, including serological, clinical and imaging based markers and algorithms have been developed in order to try and reduce the need for liver biopsy to stage fibrosis in NAFLD. Imaging modalities include magnetic resonance elastography and multi-parametric magnetic resonance imaging (MRI) as well as transient hepatic elastography (Pavlides M et al, Journal of Hepatology 2016; 64(2): 308-15; Tapper E B and Loomba R, Nat Rev Gastroenterol Hepatol 2018; 15(5): 274-82). Similarly, algorithms of varying complexity, which incorporate both serological and clinical markers, for example the Fibrosis-4 (FIB-4) score, NAFLD Fibrosis Score and Enhanced Liver Fibrosis (ELF) score are often used to help stratify patients as being at high risk of advanced liver fibrosis (Guha I N et al, Hepatology 2008; 47(2): 455-60; Angulo P et al, Hepatology 2007; 45(4): 846-5; McPherson S et al, Gut 2010; 59(9): 1265-9). However, to date, none of these approaches have been shown to be sufficiently robust to replace liver biopsy in clinical practice. In general, most of these approaches have good negative predictive value, however, sensitivity and positive predictive value are relatively poor.

The development of accurate, non-invasive markers to diagnose and stage non-alcoholic fatty liver disease (NAFLD) is therefore of high importance to reduce the need for an invasive liver biopsy and to facilitate the stratification of patients who are at the highest risk of hepatic and cardio-metabolic complications. In addition, such markers would offer the potential to track disease progression and assess treatment response in a non-invasive manner.

The present invention provides urinary biomarkers that can accurately and non-invasively diagnose and stage NAFLD.

### SUMMARY OF INVENTION

In an aspect, the invention provides a method of diagnosing non-alcoholic fatty liver disease (NAFLD) in a subject, and/or determining the stage of NAFLD in a subject diagnosed with NAFLD, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof; and
iv. using the results from (iii) to diagnose or determine the stage of non-alcoholic fatty liver disease (NAFLD) in the subject.

In another aspect, the invention provides a method of identifying a subject having an increased risk of developing liver cancer, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof;
iv. using the results from (iii) to diagnose or determine the stage of NAFLD in the subject;
wherein the patient is identified as having an increased risk of liver cancer when the stage of NAFLD is determined to be F3-F4 or F4.

In another aspect, the invention provides a method of diagnosing liver cancer in a subject, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof;
iv. using the results from (iii) to diagnose liver cancer in the subject;

In another aspect, the invention provides a method of distinguishing a subject with liver cancer from a subject with NAFLD or a healthy subject, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof; and
iv. using the results from (iii) to distinguish between subjects with liver cancer and subjects with NAFLD or healthy subjects.

In an embodiment of the above aspects, the liver cancer is hepatocellular carcinoma (HCC).

In another aspect, the invention provides a method of distinguishing a subject with NAFLD cirrhosis from a subject having alcohol related cirrhosis, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof; and
iv. using the results from (iii) to distinguish between subjects with NAFLD cirrhosis from a subject having alcohol related cirrhosis.

In another aspect, there is provided a method of treating a subject with NAFLD having advanced fibrosis and/or cirrhosis, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof; and

administering anti-NAFLD therapy to the subject if the level of the hormone or the metabolite thereof is diagnostic of cirrhosis, or the stage of NAFLD is determined as advanced fibrosis or cirrhosis.

In an embodiment, the anti-NAFLD treatment is weight loss treatment. In another embodiment, the anti-NAFLD treatment is a liver transplant. In another embodiment, the treatment may involve reducing hypertension and/or circulating lipids in a subject.

In another embodiment, the anti-NAFLD treatment is an anti-fibrotic treatment, such as nintedanib and pirfenidone.

In another aspect, there is provided a method of selecting a subject for treatment of NAFLD and/or for monitoring the progression or NAFLD and/or for assessing the efficacy of a treatment for NAFLD, wherein the method comprises:
i. providing a urine sample obtained from the subject;
ii. determining the level of at least one steroid hormone or metabolite thereof present in the sample;
iii. comparing the amount of the at least one steroid hormone or metabolite thereof detected in the sample with a reference level of the hormone or the metabolite thereof; and

selecting the subject for treatment with an anti-NAFLD therapy if the level of the hormone or the metabolite thereof is diagnostic NAFLD. The therapy administered will depend upon the stage of NAFLD.

In an embodiment of any aspect, the methods of the invention may also further comprise global analysis of steroid hormones or metabolites thereof for which the level is determined, including additional relationships and relative interactions between metabolites, herein referred to as Generalized Matrix Learning Vector Quantization (GMLVQ).

In an embodiment of any aspect of the invention, the level of any individual steroid hormone or metabolite thereof measured may be compared with a reference value.

In an embodiment, the anti-NAFLD treatment is weight loss treatment. In another embodiment, the anti-NAFLD treatment is a liver transplant.

NAFLD may be caused by or associated with one more of the following: obesity, type II diabetes, high blood pressure, high cholesterol, metabolic syndrome, hypothyroidism and hypopituitarism.

In an embodiment of any of the aspects of the invention, a subject who is diagnosed with NAFLD, or who's stage of NAFLD is determined, and/or who is identified as having an increased risk of developing liver cancer, and/or who is treated according to the invention, may be monitored after one or more of the methods of the invention are undertaken. Suitably, the monitoring may comprise ultrasound scans, for example every 6 months after a method of the invention is undertaken. Suitably, the monitoring is to determine the efficacy of any treatment. Suitably, the monitoring is for assessing NAFLD progression.

The stage of NAFLD may include any distinguishable manifestation of NAFLD. In particular the invention allows the different stages of NAFLD to be distinguished. Preferably the different stages of NAFLD are defined by the Kleiner scoring system (Kleiner et al, Hepatology 2005, Vol 41, Issue 6, 1313-1321) wherein:
F0 typically refers to a subject with an absence of liver fibrosis;
F1 typically refers to a subject with portal or perisinusoidal fibrosis,
F2 typically refers to a subject with portal/periportal and perisinusioidal fibrosis
F3 typically refers to a subject with septal or bridging liver fibrosis
F4 typically refers to a subject with cirrhosis.

Together the stage of F0-2 may be assigned to subjects having early liver fibrosis, F3-4 may be assigned to subjects having advanced liver fibrosis, and F0-3 may be assigned to subjects not having liver cirrhosis.

The method of the invention may be used to identify subjects at much earlier stages of NAFLD than current tests, and/or to monitor disease progression and/or the effectiveness or response of a subject to a particular treatment. This could also be performed in primary care settings without the need and attendant cost to attend hospital for a liver biopsy. For example, a patient may be diagnosed with NAFLD, either by the method of the invention or by other clinical parameters. A therapy or treatment plan may then be administered to the patient, and by analyzing a sample from a patient after treatment, the efficacy of the administered therapy can be assessed.

In various embodiments of the aspects of the invention, the level of at least 1, at least 2, at least 3, at least 4, at least 5, at least 6, at least 7, at least 8, at least 9, at least 10, at least 11, at least 12, at least 13, at least 14, at least 15, at least 16, at least 16, at least 17, at least 18, at least 19, at least 20, at least 21, at least 22, at least 23, at least 24, at least 25, at least 26, at least 27, at least 28, at least 29, at least 30, at least 31, or at least 32 or more steroid hormones or metabolites thereof in a urine sample are determined. For example, the level of 1, 4, 10 or 32, steroid hormones or metabolites thereof in a urine sample may be determined to perform a method of the invention.

The steroid hormone or metabolite thereof may be one or more selected from the list comprising androstendione, etiocholanolone, 11β-hydroxyandrosterone, dehydroepiandrosterone, 16α-hydroxy-dehydroepiandrosterone, pregnenetriol, pregnenediol, tetrahydro-11-dehydrocorticosterone, 5α-tetrahydro dehydrocorticosterone, tetrahydrocorticosterone, 5α-tetrahydrocorticosterone, 18-hydroxytetrahydro-11-dehydrocorticosterone, tetrahydro-11 deoxycorticosterone, tetrahydroaldosterone, pregnanediol, 3α,5α-17-hydroxypregnanolone, 17-hydroxypregnanolone, pregnanetriol, pregnanetriolone, tetrahydro-11-deoxycortisol, cortisol, 6β-hydroxy-cortisol, tetrahydrocortisol, 5α-tetrahydrocortisol, α-cortol, cortol, 11β-hydroxyetiocholanolone, cortisone, tetrahydrocortisone, α-cortolone, β-cortolone and 11-oxoetiocholanolone.

The steroid hormone or metabolite thereof, the level of which in the sample is determined in the method of the invention, may be one, two, three or all of 5α-tetrahydro-11-dehydrocorticosterone, etiocholanolone, pregnanetriol and 5α-tetrahydrocorticosterone.

In order to distinguish between a healthy subject and a subject with advanced fibrosis (with an NAFLD stage of F3-4), the level of one, two, three, four, five, six, seven, eight, nine or all of the following steroid hormones or metabolites thereof in a urine sample from the subject may be determined: 5α-tetrahydro-11-dehydrocorticosterone, 11-oxoetiocholanolone, etiocholanolone cortisone, pregnenediol, pregnanetriol, tetrahydro-11 deoxycorticosterone, 11β-hydroxyetiocholanolone, pregnanediol and 5α-tetrahydrocorticosterone. In an embodiment the level of at least 5α-tetrahydro-11-dehydrocorticosterone may be determined. In an embodiment the level of at least 5α-tetrahydro-11-dehydrocorticosterone and 11-oxoetiocholanolone may be determined. In an embodiment the level of at least 5α-tetrahydro-11-dehydrocorticosterone, 11-oxoetiocholanolone and etiocholanolone may be determined. In an embodiment the level of at least 5α-tetrahydro-11-dehydrocorticosterone, 11-oxoetiocholanolone, etiocholanolone and cortisone may be determined. In an embodiment the level of at least 5α-tetrahydro-11-dehydrocorticosterone, 11-oxoetiocholanolone, etiocholanolone, cortisone and pregnenediol may be determined. In an embodiment the level of at least 5α-tetrahydro-11-dehydrocorticosterone, 11-oxoetiocholanolone, etiocholanolone, cortisone, pregnenediol and pregnanetriol may be determined. In an embodiment the level of at least 5α-tetrahydro-11-dehydrocorticosterone, 11-oxoetiocholanolone, etiocholanolone, cortisone, pregnenediol, pregnanetriol and tetrahydro-11 deoxycorticosterone may be determined. In an embodiment the level of at least 5α-tetrahydro-11-dehydrocorticosterone, 11-oxoetiocholanolone, etiocholanolone, cortisone, pregnenediol, pregnanetriol, tetrahydro-11 deoxycorticosterone and 11β-hydroxyetiocholanolone may be determined. In an embodiment the level of at least 5α-tetrahydro-11-dehydrocorticosterone, 11-oxoetiocholanolone, etiocholanolone, cortisone, pregnenediol, pregnanetriol, tetrahydro-11 deoxycorticosterone, 11β-hydroxyetiocholanolone and pregnanediol may be determined. In an embodiment the level of at least 5α-tetrahydro-11-dehydrocorticosterone, 11-oxoetiocholanolone, etiocholanolone, cortisone, pregnenediol, pregnanetriol, tetrahydro-11 deoxycorticosterone, 11β-hydroxyetiocholanolone, pregnanediol and 5α-tetrahydrocorticosterone may be determined.

In order to distinguish between a healthy subject and a subject with cirrhosis (NAFLD stage F4) the level of one, two, three, four, five, six, seven, eight, nine or all of the following steroid hormones or metabolites thereof in a urine sample from the subject may be determined: 5α-tetrahydro-11-dehydrocorticosterone, 11-oxoetiocholanolone, etiocholanolone, cortisone, tetrahydro-11 deoxycorticosterone, pregnenediol, pregnanetriol, tetrahydrocorticosterone, pregnanediol, and 5α-tetrahydrocorticosterone. In an embodiment the level of at least 5α-tetrahydro-11-dehydrocorticosterone is determined. In an embodiment the level of at least 5α-tetrahydro-11-dehydrocorticosterone and 11-oxoetiocholanolone is determined. In an embodiment the level of at least 5α-tetrahydro-11-dehydrocorticosterone, 11-oxoetiocholanolone and etiocholanolone is determined. In an embodiment the level of at least 5α-tetrahydro-11-dehydrocorticosterone, 11-oxoetiocholanolone, etiocholanolone and cortisone is determined. In an embodiment the level of at least 5α-tetrahydro-11-dehydrocorticosterone, 11-oxoetiocholanolone, etiocholanolone, cortisone and tetrahydro-11 deoxycorticosterone is determined. In an embodiment the level of at least 5α-tetrahydro-11-dehydrocorticosterone, 11-oxoetiocholanolone, etiocholanolone, cortisone, tetrahydro-11 deoxycorticosterone and pregnenediol is determined. In an embodiment the level of at least 5α-tetrahydro-11-dehydrocorticosterone, 11-oxoetiocholanolone, etiocholanolone, cortisone, tetrahydro-11 deoxycorticosterone, pregnenediol and pregnanetriol is determined. In an embodiment the level of at least 5α-tetrahydro-11-dehydrocorticosterone, 11-oxoetiocholanolone, etiocholanolone, cortisone, tetrahydro-11 deoxycorticosterone, pregnenediol, pregnanetriol and tetrahydrocorticosterone is determined. In an embodiment the level of at least 5α-tetrahydro-11-dehydrocorticosterone, 11-oxoetiocholanolone, etiocholanolone, cortisone, tetrahydro-11 deoxycorticosterone, pregnenediol, pregnanetriol, tetrahydrocorticosterone and pregnanediol is determined. In an embodiment the level of at least 5α-tetrahydro dehydrocorticosterone, 11-oxoetiocholanolone, etiocholanolone, cortisone, tetrahydro-11 deoxycorticosterone, pregnenediol, pregnanetriol, tetrahydrocorticosterone, pregnanediol, and 5α-tetrahydrocorticosterone is determined.

In order to distinguish between a subject with early stage liver fibrosis, (NAFLD stage of F0 to F2), and a subject with advanced fibrosis (NAFLD stage of F3 to F4), the level of one, two, three, four, five, six, seven, eight, nine or all of the following steroid hormones or metabolites thereof in a urine sample from the subject may be determined: etiocholanolone, dehydroepiandrosterone, 5α-tetrahydro-11-dehydrocorticosterone, androstendione, 5α-tetrahydrocorticosterone, pregnenetriol tetrahydro-11 deoxycorticosterone, tetrahydroaldosterone, cortisone and 11-oxoetiocholanolone. In an embodiment the level of at least etiocholanolone is determined. In an embodiment the level of at least etiocholanolone and dehydroepiandrosterone is determined. In an embodiment the level of at least etiocholanolone, dehydroepiandrosterone and 5α-tetrahydro-11-dehydrocorticosterone is determined. In an embodiment the level of at least etiocholanolone, dehydroepiandrosterone, 5α-tetrahydro-11-dehydrocorticosterone and androstendione is determined. In an embodiment the level of at least etiocholanolone, dehydroepiandrosterone, 5α-tetrahydro-11-dehydrocorticosterone, androstendione and 5α-tetrahydrocorticosterone is determined. In an embodiment the level of at least etiocholanolone, dehydroepiandrosterone, 5α-tetrahydro-11-dehydrocorticosterone, androstendione, 5α-tetrahydrocorticosterone and pregnenetriol is determined. In an embodiment the level of at least etiocholanolone, dehydroepiandrosterone, 5α-tetrahydro-11-dehydrocorticosterone, androstendione, 5α-tetrahydrocorticosterone, pregnenetriol and tetrahydro-11 deoxycorticosterone is determined. In an embodiment the level of at least etiocholanolone, dehydroepiandrosterone, 5α-tetrahydro-11-dehydrocorticosterone, androstendione, 5α-tetrahydrocorticosterone, pregnenetriol, tetrahydro-11 deoxycorticosterone and tetrahydroaldosterone is determined. In an embodiment the level of at least etiocholanolone, dehydroepiandrosterone, 5α-tetrahydro-11-dehydrocorticosterone, androstendione, 5α-tetrahydrocorticosterone, pregnenetriol, tetrahydro-11 deoxycorticosterone, tetrahydroaldosterone and cortisone is determined. In an embodiment the level of at least etiocholanolone, dehydroepiandrosterone, 5α-tetrahydro-11-dehydrocorticosterone, androstendione, 5α-tetrahydrocorticosterone, pregnenetriol, tetrahydro-11 deoxycorticosterone, tetrahydroaldosterone, cortisone and 11-oxoetiocholanolone is determined.

In order to distinguish between a subject with an NAFLD stage of F0 to F3 and a subject with cirrhosis (NAFLD stage F4), the level of one, two, three, four, five, six, seven, eight, nine or all of the following steroid hormones or metabolites thereof in a urine sample from the subject may be determined: etiocholanolone, tetrahydrocorticosterone, 5α-tetrahydro-11-dehydrocorticosterone, tetrahydro-11 deoxycorticosterone, tetrahydrocortisol, dehydroepiandrosterone, androstendione, tetrahydrocortisone, pregnenetriol and 5α-tetrahydrocorticosterone. In an embodiment the level of at least etiocholanolone is determined. In an embodiment the level of at least etiocholanolone and tetrahydrocorticosterone is determined. In an embodiment the level of at least etiocholanolone, tetrahydrocorticosterone and 5α-tetrahydro-11-dehydrocorticosterone is determined. In an embodiment the level of at least etiocholanolone, tetrahydrocorticosterone, 5α-tetrahydro-11-dehydrocorticosterone and tetrahydro-11 deoxycorticosterone is determined. In an embodiment the level of at least etiocholanolone, tetrahydrocorticosterone, 5α-tetrahydro-11-dehydrocorticosterone, tetrahydro-11 deoxycorticosterone and dehydroepiandrosterone is determined. In an embodiment the level of at least etiocholanolone, tetrahydrocorticosterone, 5α-tetrahydro-11-dehydrocorticosterone, tetrahydro-11 deoxycorticosterone, dehydroepiandrosterone and androstendione is determined. In an embodiment the level of at least etiocholanolone, Tetrahydrocorticosterone, 5α-tetrahydro-11-dehydrocorticosterone, tetrahydro-11 deoxycorticosterone, dehydroepiandrosterone, androstendione and tetrahydrocortisone is determined. In an embodiment the level of at least etiocholanolone, tetrahydrocorticosterone, 5α-tetrahydro-11-dehydrocorticosterone, tetrahydro-11 deoxycorticosterone, dehydroepiandrosterone, androstendione, tetrahydrocortisone and tetrahydrocortisol is determined. In an embodiment the level of at least etiocholanolone, tetrahydrocorticosterone, 5α-tetrahydro-11-dehydrocorticosterone, tetrahydro-11-deoxycorticosterone, dehydroepiandrosterone, androstendione, tetrahydrocortisone, tetrahydrocortisol and pregnenetriol are determined. In an embodiment the level of at least etiocholanolone, tetrahydrocorticosterone, 5α-tetrahydro-11-dehydrocorticosterone, tetrahydro-11 deoxycorticosterone, dehydroepiandrosterone, androstendione, tetrahydrocortisone, tetrahydrocortisol, pregnenetriol and 5α-tetrahydrocorticosterone is determined.

In an embodiment of any aspect of the invention, the level of at least seven steroid hormones or metabolites thereof in a urine sample from the subject may be determined. Suitably, the at least seven steroid hormones or metabolites thereof may be selected from androstendione, etiocholanolone, 11β-hydroxyandrosterone, dehydroepiandrosterone, 16α-hydroxy-dehydroepiandrosterone, pregnenetriol, pregnenediol, tetrahydro-11-dehydrocorticosterone, 5α-tetrahydro-11-dehydrocorticosterone, tetrahydrocorticosterone, 5α-tetrahydrocorticosterone, 18-hydroxytetrahydro-11-dehydrocorticosterone, tetrahydro-11 deoxycorticosterone, tetrahydroaldosterone, pregnanediol, 3α,5α-17-hydroxypregnanolone, 17-hydroxypregnanolone, pregnanetriol, pregnanetriolone, tetrahydro-11-deoxycortisol, cortisol, 6β-hydroxy-cortisol, tetrahydrocortisol, 5α-tetrahydrocortisol, α-cortol, cortol, 11β-hydroxyetiocholanolone, cortisone, tetrahydrocortisone, α-cortolone, cortolone and 11-oxoetiocholanolone.

In an embodiment of any aspect of the invention, the subject may be given a prognosis based on the stage of NAFLD determined.

The step of determining the level of at least one steroid hormone or metabolite thereof in the urine sample of any method of the invention may comprise the steps of:
a. extracting free and conjugated steroid hormones or metabolites thereof from the urine sample
b. quantifying the steroid hormones or metabolites thereof in the extraction.

The step of determining the level of at least one steroid hormone or metabolite thereof in the urine sample of any method of the invention may comprise the steps of:
a. extracting free and conjugated steroid hormones or metabolites thereof, for example by solid phase extraction, from the urine sample;
b. hydrolysing the extracted conjugated steroid hormones or metabolites thereof, for example by enzymatic hydrolysis;
c. re-extracting the hydrolysed conjugates of steroid hormones or metabolites thereof, for example using solid phase extraction;
d. performing chemical derivatization on the free and hydrolysed conjugates of steroid hormones or metabolites thereof, to form ethers;
e. performing liquid-liquid extraction; and
f. quantifying the steroid hormones or metabolites thereof in the extraction, for example by using GC/MS (Gas Chromatography/Mass Spectrometry).

The method of the invention may be performed using high-throughput liquid chromatography/tandem mass spectrometry.

The method of the invention may further comprise the step of urinary creatinine correction. This may allow the results to be adjusted for differing times and durations of collection of the urine sample.

The methods of the invention may further comprise the step of calculating precursor metabolite to product metabolite ratios.

In an embodiment, the level and/or presence of particular steroid hormones or metabolites thereof may be determined in a simple point of care test, such as with a colorimetric indicator on a spot test or lateral flow device.

Samples may be analysed by means of a biochip. Biochips generally comprise solid substrates and have a generally planar surface to which a capture reagent (also called an adsorbent or affinity reagent) is attached. Frequently, the surface of a biochip comprises a plurality of addressable locations, each of which has the capture reagent bound there.

The term ‘urine sample’ defined herein includes any sample of urine from a subject, ranging from about 0.01 mL, or about 0.5 mL, or about 1 mL to about 3 mL. The sample may be fresh, be stored for up to 1 hour, up to 2 hours, up to 4 hours, up to 8 hours, up to 12 hours, up to 16 hours, or up to 24 hours at 4° C., or be stored indefinitely at −80° C. before performing a method of the invention. Preferably the urine sampled is a single urine sample, taken at any time of day.

The step of obtaining the sample may not form part of the invention.

The method of the invention may be carried out in vitro.

The subject may be a mammal and is preferably a human, but may alternatively be a monkey, ape, cat, dog, cow, horse, rabbit or rodent.

The reference value may be the level of the steroid hormone or metabolite thereof in a subject with a known stage of NAFLD with which the sample is being compared, or from a healthy subject. The reference value may be the level of the steroid hormone or a metabolite thereof from the subject at an earlier time, for example before treatment commenced.

In an embodiment, the subject's age, BMI, the presence and/or level of serological markers or any combination thereof may be used when performing a method of the invention.

Thus, any aspect of the invention may further comprise measuring the level of one or more serological markers in a subject. Suitably, the level of the one or more serological markers is measured from a blood sample obtained from the subject. The skilled person will understand that there are various techniques at their disposal to measure the level of the one or more serological markers. Suitable serological markers may give an indication of liver function. Suitable serological markers may comprise or consist of one or more of alanine aminotransferase (ALT), aspartate aminotransferase (AST), and haemoglobin Act (HbA1c).

Early diagnosis of NAFLD or without and early determination of advanced fibrosis or cirrhosis in a subject diagnosed with NAFLD, and early intervention in each of these circumstances, could prevent early death of a subject.

The method of the invention may also be used to monitor NAFLD stage progression, and/or to monitor the efficacy of treatments and/or preventive regimes administered to a subject. This may be achieved by analysing samples taken from a subject at various time points following initial diagnosis and monitoring the changes in the level of steroid hormone or metabolites thereof in subsequent urine sample. In this case reference levels may include the initial levels/profile of the steroid hormones or metabolites thereof, or the levels or profile of the steroid hormones or metabolites thereof in the subject when they were last tested, or both.

The invention may further provide a panel of biomarkers comprising one or more of androstendione, etiocholanolone, 11β-hydroxyandrosterone, dehydroepiandrosterone, 16α-hydroxy-dehydroepiandrosterone, pregnenetriol, pregnenediol, tetrahydro-11-dehydrocorticosterone, 5α-tetrahydro dehydrocorticosterone, tetrahydrocorticosterone, 5α-tetrahydrocorticosterone, 18-hydroxytetrahydro-11-dehydrocorticosterone, tetrahydro-11 deoxycorticosterone, tetrahydroaldosterone, pregnanediol, 3α,5α-17-hydroxypregnanolone, 17-hydroxypregnanolone, pregnanetriol, pregnanetriolone, tetrahydro-11-deoxycortisol, cortisol, 6β-hydroxy-cortisol, tetrahydrocortisol, 5α-tetrahydrocortisol, α-cortol, β-cortol, 11β-hydroxyetiocholanolone, cortisone, tetrahydrocortisone, α-cortolone, β-cortolone and 11-oxoetiocholanolone. The panel may comprise one, two, three or all of 5α-tetrahydro-11-dehydrocorticosterone, etiocholanolone, pregnanetriol and 5α-tetrahydrocorticosterone. The panel may be used to diagnose NAFLD in a subject or to determine the stage of NAFLD status in a subject.

The skilled person will appreciate that preferred features of any one embodiment and/or aspect of the invention may be applied to all other embodiments and/or aspects of the invention.

### BRIEF DESCRIPTION OF THE FIGURES

FIG. 1: shows the results of the determination of the total glucocorticoid metabolite level, and 11β-hydroxysteroid dehydrogenase type 1 and 5α-reductase activity in healthy controls and subjects with early or late stages of liver disease (NAFLD). Statistical analysis was performed on log transformed steroid values or ratios. Data shown: mean±SD. 2 and 4 data points not shown in FIG. 1A and FIG. 1B respectively for graphical purposes. Both 11β-hydroxysteroid dehydrogenase type 1 (FIG. 1A) and 5α-reductase (FIG. 1B) activity are increased in subjects with NAFLD with advanced fibrosis, although not in those with mild disease when compared to healthy controls. Total glucocorticoid metabolite production was not different across the spectrum of NAFLD or in comparison with healthy controls (FIG. 1C) (**** p<0.0001, * p<0.05).

FIG. 2: shows GMLVQ analysis of subjects with NAFLD compared to healthy controls. Numerical values are given for each individual steroid metabolite (Table 3). FIG. 2A is a two-dimensional visualization of steroid data obtained by projection of the z-score transformed and log-scaled excretion values onto the first and second eigenvector of the relevance matrix. Prototypical representatives of disease classes (healthy controls and NAFLD fibrosis stages) using z-score transformed log-scaled steroid excretion values are shown in FIG. 2B. FIG. 2C shows diagonal elements of the relevance matrix (normalized to sum 1), indicating the importance of individual steroids in the GMLVQ classifier.

FIG. 3: is a demonstration of GMLVQ and ROC AUC analysis which provides improved separation between different stages of liver disease compared to conventional separation methods. FIG. 3A shows that GMLVQ′ analysis permits very good separation between early and advanced fibrosis (F0-2 vs. F3-4) in patients with NAFLD. ROC AUC analysis is presented in FIG. 3B in comparison with FIB-4. FIG. 3C shows that the performance of GMLVQ to identify subjects with cirrhosis (F0-3 vs. F4) is also very good, with ROC AUC analysis demonstrating in FIG. 3B significant improvement in diagnostic ability when compared to NAFLD fibrosis score.

FIG. 4: demonstrates that GMLVQ* analysis has excellent potential utility as a screening tool to identify individuals with advanced NAFLD fibrosis within the general population. FIG. 4A shows there was excellent separation between healthy controls and those with advanced NAFLD fibrosis with the corresponding ROC AUC analysis FIG. 4B. The performance of GMLVQ* to identify patients with NAFLD cirrhosis in the general population (healthy control vs. F4) is excellent with perfect separation (FIGS. 4C and D).

FIG. 5: demonstrates of the ability of GMLVQ and GMLVQ* to identify advanced stages of liver diseases. Identification of advanced stages of NAFLD fibrosis (F3-4) (FIG. 5A) and cirrhosis (F4) (FIG. 5B) can be refined to a panel of approximately 10 specific steroid metabolites (GMLVQ-10*) without significant reduction in diagnostic performance.

FIG. 6: shows that GMLVQ′ analysis permits very good separation between NAFLD cirrhosis and alcohol related cirrhosis (a). ROC AUC analysis demonstrates potential clinical utility in determining underlying cirrhosis aetiology (b).

FIG. 7: demonstrates a good correlation between the levels of key discriminatory steroids used in the GMLVQ analysis when they are measured by GC/MS or LC MS/MS (a and b). The performance of the GMLVQ analysis to discriminate F0-2 vs. F3-4 is not significantly different when steroid metabolites are measured either by GC MS (c) or LC MS/MS (d). [The analysis in panel c and d was performed on a small subset of patients with NAFLD (n=75) in comparison with the full published data series (ref) and this is reflected in the AUC ROC values.]

### MATERIALS AND METHODS

Clinical data and urine samples (spot or 24 hour collections) were collected from 275 subjects including 121 with NAFLD, 106 from healthy controls without known liver disease and 48 with alcohol-related cirrhosis. Detailed demographic information is presented in Table 1. All patients with NAFLD had liver biopsy staging performed, except in 6 patients where a diagnosis of cirrhosis was made using established clinical criteria (clinical examination, platelets and liver function blood tests, imaging, elastography). Determination of healthy control status was established by review of medical history and the absence of any known liver disease. Healthy control subjects with abnormal liver chemistry or with elevated non-invasive serum fibrosis assessments (see below) were excluded from the analysis. Where data in individual subjects was available, scores for non-invasive markers of liver fibrosis were calculated. These were defined as follows:

APRI(AST to Platelet Ratio Index)=AST (IU/L)/(upper limit of normal)/platelet count(×10⁹/L)×100

FIB-4(Fibrosis-4 score)=age×AST (IU/L)/platelet count(×10⁹/L)×√ALT (IU/L)

AST/ALT ratio=AST (IU/L)/ALT (IU/L)

NAFLD fibrosis score=−1.675+0.037×age (years)+0.094×BMI (kg/m²)+1.13×Impaired fasting glucose or T2D(yes=1,no=0)+0.99×AST/ALT ratio−0.013×platelet count(×10⁹/L)−0.66×albumin(g/dL)

BARD score=sum(BMI>28 kg/m²=1,AST/ALT ratio>0.8=2,T2D=1)

Histological Liver Staging of NAFLD

Liver biopsies were performed as part of routine clinical care in patients with NAFLD. NAFLD Activity Score (NAS) (including the individual components of lobular, inflammation, steatosis, hepatocyte ballooning and fibrosis) as well as NAFLD fibrosis stage (F0-F4) was assessed by the Kleiner scoring system. F0 represents the absence of fibrosis, F1 portal or perisinusoidal fibrosis, F2 portal/periportal and perisinusioidal fibrosis, F3 septal or bridging fibrosis and F4 cirrhosis.

Urinary Steroid Metabolites Analysis Using GC-MS

Urine samples were collected and stored at −80° C. Measurement of urinary steroid metabolites was undertaken using gas chromatography/mass spectrometry (GC/MS) as has been previously reported (Krone et al, The Journal of Steroid Biochemistry and Molecular Biology 2010; 121(3-5): 496-504).

In brief, free and conjugated steroids were extracted from 1 mL of urine via a 5-step extraction method. Solid-phase extraction of free and conjugated steroids was performed. Steroid conjugates underwent enzymatic hydrolysis followed by solid-phase re-extraction of steroids, chemical derivatization to form ethers, and finally liquid-liquid extraction. GC/MS was undertaken on an Agilent 5973 MSD single quadrupole gas chromatography mass spectrometer (Agilent, Santa Clara, USA) instrument allowing quantification of up to 32 steroid metabolites, with representation of major steroids and their metabolites from all the adrenally derived steroid hormone classes (androgens, glucocorticoids and mineralocorticoids (Table 3). Steroids were identified in SIM (single ion monitoring mode) and quantified relative to authentic reference standards.

For each urine sample a urinary creatinine correction was made in an attempt to adjust for differing times and durations of collection as urinary creatinine is excreted at a relatively constant rate and is widely used as a corrective factor in the analysis of urine metabolites (Tsikas et al, J Chromoatogr B Analyt Technol Biomed Life Sci 2010; 878(27): 2582-92). These data were expressed as μg steroid/g urinary creatinine. A separate analysis of uncorrected data expressed as μg steroid/1000 mL urine was also undertaken.

Measurement of individual steroid hormone concentrations and their metabolites permitted assessment of individual steroid metabolic pathways based upon the analysis of ‘precursor metabolite to product metabolite’ ratios. This approach allows the assessment of specific enzymatic activities. All individual steroid data was log transformed (Log 10) prior to analysis. Product to pre-cursor metabolite ratios investigating specific pathways of glucocorticoid metabolism were calculated as follows:
Total Cortisol (F) Metabolites=6β-hydroxy-cortisol+tetrahydrocortisol (THF)+5α-tetrahydrocortisol (5αTHF)+α-cortol+β-cortol+11β-hydroxyetiocholanolone+cortisone (E)+tetrahydrocortisone (THE)+α-cortolone+β-cortolone+11-oxoetiocholanolone
11β-HSD1 activity=(THF+5αTHF)/THE
A-ring reductase activity=5αTHF/THF

Urinary Creatinine Assay

Urinary creatinine measurement was performed using the QuantiChrom™ Creatinine Assay Kit (DICT-500, Universal Biologicals, UK). 54 of either standard (50 mg/dL) or urine were mixed with 2004 of working reagent in a 96-well plate. Optical density (OD) was read at 0 min and 5 min at an absorbance of 490 nm on a VersaMax Plate Reader (Molecular Devices, UK) and the creatinine concentration (mg/dL) was calculated for each urine sample in duplicate as per the manufacturer guidance. A mean creatinine value (mg/dL) was calculated from a minimum of 2 independent assays.

Generalized Matrix Learning Vector Quantization (GMLVQ) Computational Analysis

Learning Vector Quantization (LVQ) is a machine learning technique that extracts typical class representatives or prototypes from training data (Biehl et al, Wiley Interdiscip Rev Cogn Sci; 2016; 7(2): 92-111. For our application this translated to one typical steroid profile per disease stage. These prototypes can be used to classify a steroid profile with unknown disease stage: the most probable disease stage is determined by selecting the class of the prototype that is most similar to the new profile. The dis-similarity of a given steroid profile and a prototype is defined by a distance measure, for example the conventional Euclidean distance. In Generalized Matrix Learning Vector Quantization (GMLVQ) (Schneider et al, Neural Comput 2009; 21(12): 3532-61) however, the distance metric itself is adaptive and optimized together with the prototypes in the same data driven training process. This metric is defined through a matrix of adaptive parameters, termed the relevance matrix. Its diagonal elements quantify the importance of individual steroids in the classification scheme.

GMLVQ analysis of GC-MS data was performed in all subjects who provided a spot urine sample using a panel of 32 steroids. Steroid data was log transformed (Log 10) before undergoing standardisation by z-score transform prior to GMLVQ analysis. Missing values were treated along the lines of the NaN-LVQ (Not a Number-Learning Vector Quantization) prescription, ignoring them in the computation of the corresponding distances (Ghosh et al, European Symposium on Artificial Neural Networks, Computational Intelligence and Machine Learning 2017; i6doc.com publishing: 199-204).

Feature selection was used to refine the model to investigate the performance of a reduced number of steroids. The top 10 most relevant steroids were identified from the relevance matrix to reduce the steroid number from 32 to 10. Following this, a backwards elimination ‘greedy search’ strategy was employed to reduce the number of steroids from 10 to 2 sequentially which involved re-training the GMLVQ system each time the least relevant steroid was removed.

Due to the number of subjects in the cohort, repeated random sub-sampling validation (Hastie et al, The Elements of Statistical Learning Springer Series in Statistics 2017) was applied to divide the dataset into training and validation sets in order to evaluate GMLVQ performance. The process was repeated to produce 200 results, each one corresponding to a division of 90% for training and 10% for validation. The randomized sets were stratified in the sense that both training and validation sets contained at least one example from each class.

Receiver operating characteristics (ROC) (Hastie et al, The Elements of Statistical Learning Springer Series in Statistics 2017; T.F An Introduction to ROC Analysis. Pattern Recognition Letters 2006; 27: 861-74) and area under curve (AUC) of the ROC curve was used as the primary performance metric to compare newly generated models and various alternative established non-invasive scores for liver fibrosis. Bootstrapping (Hastie et al, The Elements of Statistical Learning Springer Series in Statistics 2017) was used to calculate 95% confidence intervals for the mean ROC values and mean feature relevances. 10,000 bootstrap samples were taken from the 200 validation results. Mean values per sample were calculated and the borders of the centre 95% values were used to provide the confidence interval.

Statistical Analysis

Steroid metabolite ratio data is graphically represented as mean and standard error of the mean using GraphPad Prism version 7.02 (GraphPad Software, California). Individual steroid data and steroid ratios were compared between controls, early fibrosis and advanced fibrosis groups using the Kruskal-Wallis non-parametric test and pair-wise multiple comparisons between groups were undertaken using Dunn's post hoc test. Significance was determined as p<0.05.

### EXAMPLES

### Example 1

275 individuals were recruited into the study. Demographic details as well as biochemical and histological assessment are presented in Table 1.

TABLE 1
Demographic details of 227 subjects: 106 controls and
121 individuals with biopsy-proven NAFLD stratified by
fibrosis stage (F0-2 vs. F3-4). Data expressed are mean ±
standard deviation (unless otherwise stated).
(*p < 0.05 vs. control; ^{§}p < 0.05 vs. F0-2)

| | | | | p- |
|---|---|---|---|---|
| | Control | F0-2 | F3-4 | value |
| N (m/f) (males, | 106 (41/65) | 39 (20/19) | 82 (39/43) | 0.29 |
| %) | (38.7) | (51.3) | (47.6) | |
| Age | 55.5 ± 11.1 | 45.6 ± 12.0* | 61.8 ± 10.8*^{§} | <0.0001 |
| BMI | 30.7 ± 5.8 | 38.5 ± 7.0* | 33.7 ± 5.8*^{§} | <0.0001 |
| Proportion | 3.8 | 30.8* | 63.4*^{§} | <0.0001 |
| with Type 2 | | | | |
| Diabetes, % | | | | |
| HbA1c, mmol/ | 38.6 ± 10.4 | 40.8 ± 8.2 | 50.0 ± 13.5*^{§} | <0.0001 |
| mol | | | | |
| Platelets, 10⁹/L | n/a | 242.5 ± 64.2 | 183.9 ± 67.0^{§} | <0.0001 |
| ALT, IU/L | 13.2 ± 8.7 | 63.4 ± 51.4* | 49.9 ± 36.9* | <0.0001 |
| AST, IU/L | n/a | 34.4 ± 22.0 | 49.1 ± 31.8^{§} | 0.0006 |
| Fib-4 Score | n/a | 0.931 ± 0.7 | 2.61 ± 1.7^{§} | <0.0001 |
| NAFLD | n/a | 1.9 ± 1.2 | 3.8 ± 1.6^{§} | <0.0001 |
| Fibrosis Score | | | | |
| NAS Score | n/a | 4.0 ± 1.7 | 4.7 ± 1.3^{§} | 0.029 |
| (0-8) | | | | |
| Proportion with | n/a | 42.1 | 63.1 | 0.07 |
| NASH (NAS | | | | |
| Score > 4, %) | | | | |

Increased 11β-Hydroxysteroid Dehydrogenase Type 1 and 5α-Reductase Activity in Patients with Advanced NAFLD.

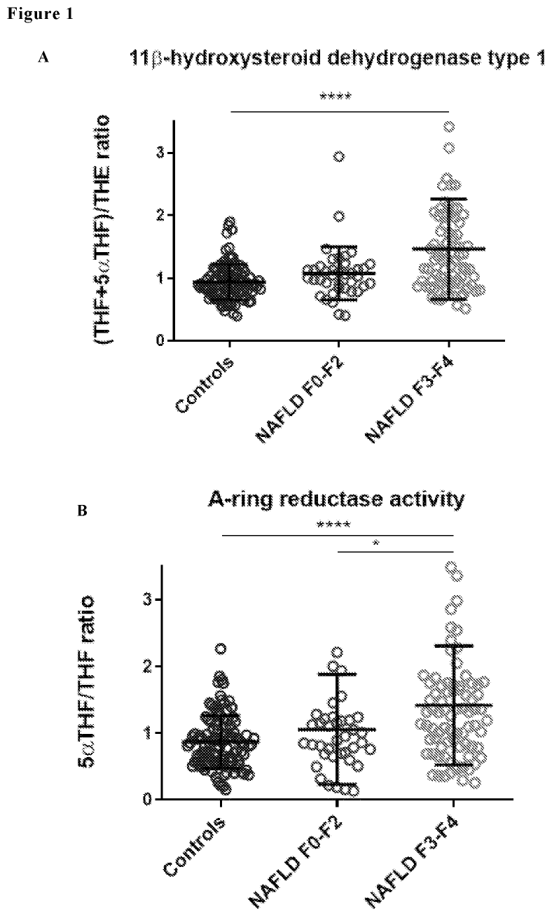

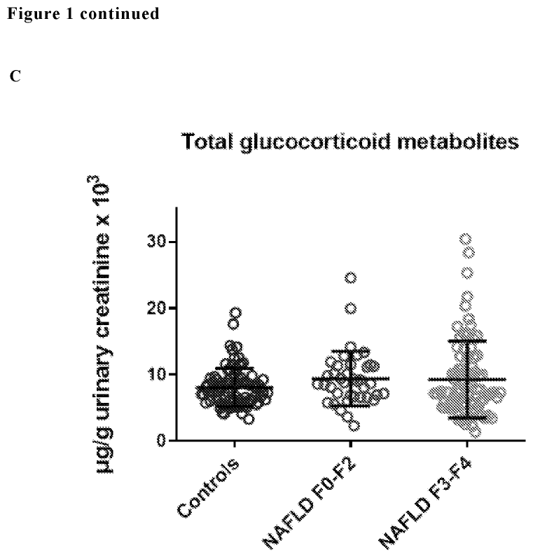

Data for specific steroid metabolites and ratios indicative of specific enzyme activity are presented in Table 2. Previous studies in small numbers of patients (often without liver biopsy) have identified specific changes in urinary steroid metabolites ratio^{17,18}. In this cohort, the (THF+5αTHF)/THE ratio reflecting 11β-HSD1 activity was increased, consistent with enhanced cortisol regeneration, in patients with advanced NAFLD (FIG. 1A), though not in those with mild disease (F0-2). In parallel, an increase in 5α-reductase activity was observed, which would enhance cortisol clearance (FIG. 1B). There was no change in total glucocorticoid metabolite production (FIG. 1C).

TABLE 2
Urinary corticosteroid metabolite analysis performed by GC/MS on spot
urine samples from 106 controls subjects and 121 with NAFLD stratified by fibrosis
stage. (THE = tetrahydrocortisone, THF = tetrahydrocortisol, UFF = urinary free
cortisol, UFE = urinary free cortisone, 11OH-androst = 11hydroxyandrosterone,
11OH-etio = 11hydroxyetiocholanolone, 11oxo-etio = 11oxo-etiocholanolone, Total
glucocorticoid metabolites = cortisol + 6β-OH-Cortisol + THF + 5αTHF + α-cortol + β-
cortol + 11b-OH-ETIO + cortisone + THE + α-cortolone + β-cortolone + 11-oxo-etio, Fm =
cortisol + THF + 5αTHF + α-cortol + β-cortol, Em = cortisone + THE + α-cortolone + β-
cortolone). Statistical analysis was performed on log transformed steroid values or
ratios, *p < 0.05 vs. control; ^{§}p < 0.05 vs. F0-2.

| | | |
|---|---|---|
| | NAFLD | NAFLD |
| Control | F0-2 | F3-4 |

Corticosteroid metabolites, μg/g urinary creatinine (mean ± SEM)

| | | | |
|---|---|---|---|
| Androstendione | 959 ± 53 | 1787 ± 328 | 835 ± 81*^{§} |
| Etiocholanolone | 918 ± 56 | 1029 ± 121 | 466 ± 55*^{§} |
| 11β-hydroxyandrosterone (11OH- | 442 ± 21 | 630 ± 87 | 642 ± 46* |
| androst) | | | |
| Dehydroepiandrosterone (DHEA) | 249 ± 44 | 428 ± 103 | 139 ± 39*^{§} |
| 16α-hydroxy-dehydroepiandrosterone | 287 ± 31 | 347 ± 54 | 387 ± 49 |
| Pregnenetriol (5-PT) | 156 ± 13 | 284 ± 37* | 165 ± 20^{§} |
| Pregnenediol (5-PD) | | | |
| Tetrahydro-11- | 97 ± 7 | 101 ± 9 | 83 ± 9*^{§} |
| Dehydrocorticosterone (THA) | | | |
| 5α-tetrahydro-11- | 87 ± 3 | 80 ± 7 | 60 ± 7*^{§} |
| Dehydrocorticosterone (5αTHA) | | | |
| Tetrahydrocorticosterone (THB) | 101 ± 8 | 107 ± 11 | 98 ± 14^{§} |
| 5α-tetrahydrocorticosterone (5αTHB) | 219 ± 14 | 296 ± 31 | 217 ± 24^{§} |
| 18-hydroxytetrahydro-11- | 44 ± 3. | 46 ± 4 | 57 ± 5 |
| Dehydrocorticosterone | | | |
| Tetrahydro-11 deoxycorticosterone | 14 ± 1 | 12 ± 1 | 9 ± 1*^{§} |
| (TH-DOC) | | | |
| Tetrahydroaldosterone (3a5bThaldo) | 30 ± 2 | 26 ± 2 | 43.7 ± 4*^{§} |
| Pregnanediol (PD) | 161 ± 16 | 132 ± 17 | 128 ± 29* |
| 3α,5α-17-hydroxypregnanolone | 9 ± 1 | 15 ± 2 | 12 ± 1 |
| 17-hydroxypregnanolone | 85 ± 7 | 89 ± 9 | 79 ± 11^{§} |
| Pregnanetriol (PT) | 323 ± 16 | 316 ± 24 | 234 ± 19*^{§} |
| Pregnanetriolone | 25 ± 6 | 16 ± 2 | 15 ± 2^{§} |
| Tetrahydro-11-deoxycortisol (THS) | 68 ± 5 | 65 ± 7 | 80 ± 10 |
| Cortisol | 56 ± 6 | 87 ± 14* | 139 ± 16*^{§} |
| 6-hydroxy-cortisol | 94 ± 5 | 122 ± 19 | 161 ± 16* |
| Tetrahydrocortisol (THF) | 1389 ± 69 | 1583 ± 130 | 1512 ± 145 |
| 5-tetrahydrocortisol (5αTHF) | 1114 ± 54 | 1669 ± 211 | 1682 ± 126* |
| α-cortol | 269 ± 14 | 334 ± 23* | 406 ± 35* |
| β-cortol | 380 ± 20 | 381 ± 25 | 434 ± 28 |
| 11β-hydroxyetiocholanolone (11OH- | 228 ± 16 | 122 ± 16* | 123 ± 13* |
| etio) | | | |
| Cortisone | 78 ± 4 | 104 ± 14 | 191 ± 17*^{§} |
| Tetrahydrocortisone | 2835 ± 130 | 3021 ± 223 | 2607 ± 225^{§} |
| α-cortolone | 1130 ± 52 | 1297 ± 79 | 1282 ± 86 |
| β-cortolone | 551 ± 24 | 526 ± 30 | 620 ± 44 |
| 11-oxoetiocholanolone (11oxo-etio) | 296 ± 19 | 168 ± 16* | 123 ± 12* |
| Total glucocorticoid metabolites | 8072 ± 306 | 9415 ± 656 | 9282 ± 638 |
| Total cortisol metabolites (Fm) | 3208 ± 133 | 4054 ± 351 | 4174 ± 308 |
| Total cortisone metabolites (Em) | 4595 ± 192 | 4949 ± 311 | 4701 ± 336 |

Corticosteroid metabolite ratios (mean ± SEM)

| | | | |
|---|---|---|---|
| UFF/UFE (cortisol/cortisone) | 0.7 ± 0 | 0.8 ± 0.1* | 0.7 ± 0^{§} |
| (THF + 5αTHF)/THE | 0.9 ± 0 | 1.1 ± 0.1 | 1.5 ± 0.1* |
| Cortols/cortolones ((α-cortol + β- | 0.4 ± 0 | 0.4 ± 0 | 0.5 ± 0*^{§} |
| cortol)/(α-cortolone + β-cortolone)) | | | |
| (11OH-androst + 11OH-etio)/11oxo-etio | 2.9 ± 0.1 | 5.7 ± 0.8* | 9.3 ± 0.8*^{§} |
| 5αTHF/THF | 0.9 ± 0 | 1.1 ± 0.1 | 1.4 ± 0.1*^{§} |
| Androsterone/Etiocholanolone | 1.2 ± 0.1 | 1.8 ± 0.2* | 2.5 ± 0.2* |

TABLE 3
Chemical names of individual steroid metabolites.

| No. | Common name | Chemical name |
|---|---|---|
| 1 | Androstendione | 5α-androstan-3α-ol-17-one |
| 2 | Etiocholanolone | 5β-androstan-3α-ol-17-one |
| 3 | 11β-hydroxyandrosterone | 5α-androstane-3α,11β-diol-17-one |
| 4 | Dehydroepiandrosterone | 5-androsten-3β-ol-17-one |
| 5 | 16α-hydroxy- | 5-androstene-3β,16α-diol-17-one |
| | dehydroepiandrosterone | |
| 6 | Pregnenetriol | 5-pregnene-3β,17,20-triol |
| 7 | Pregnenediol | 5-pregnene-3β,20α-diol and |
| | | 5,17,(20)-pregnadien-3-ol |
| 8 | Tetrahydro-11- | 5α-pregnane-3α,21-diol,11,20- |
| | dehydrocorticosterone | dione |
| 9 | 5α-tetrahydro-11- | |
| | dehydrocorticosterone | |
| 10 | Tetrahydrocorticosterone | 5β-pregnane-3α,11β,21-triol-20- |
| | | one |
| 11 | 5α- | 5α-pregnane-3α,11β,21-triol-20- |
| | tetrahydrocorticosterone | one |
| 12 | 18 -hydroxytetrahydro-11- | 5β-pregnane-3α,3,18,21- |
| | dehydrocorticosterone | trihydroxy-11,20-dione |
| 13 | Tetrahydro-11 | 5β-pregnane-3α,21-diol-20-one |
| | deoxycorticosterone | |
| 14 | Tetrahydroaldosterone | 5β-pregnane-3α,11β,21-triol-20- |
| | | one-18-al |
| 15 | Pregnanediol | 5β-pregnane-3α,20α-diol |
| 16 | 3α,5α-17- | 5α-pregnane-3α,17α-diol-20-one |
| | hydroxypregnanolone | |
| 17 | 17-hydroxypregnanolone | 5β-pregnane-3α,17α-diol-20-one |
| 18 | Pregnanetriol | 5β-pregnane-3α,17α,20α-triol |
| 19 | Pregnanetriolone | 5β-pregnane-3,17,20α-triol-11-one |
| 20 | Tetrahydro-11- | 5β-pregnane-3α,17,21-triol-20-one |
| | deoxycortisol | |
| 21 | Cortisol | 4-pregnene-11β,17,21-triol-3,20- |
| | | dione |
| 22 | 6β-hydroxy-cortisol | 4-pregnene-6β,11β,17,21-tetrol- |
| | | 3,20-dione |
| 23 | Tetrahydrocortisol | 5β-pregnane-3α,11β,17,21-tetrol- |
| | | 20 one |
| 24 | 5α-tetrahydrocortisol | 5α-pregnane-3α,11β,17,21-tetrol- |
| | | 20-one |
| 25 | α-cortol | 5β-pregnan-3α,11β,17,20α,21- |
| | | pentol |
| 26 | β-cortol | 5β-pregnan-3α,11β,17,20β,21- |
| | | pentol |
| 27 | 11β- | 5-androstane-3α,11β-diol-17-one |
| | hydroxyetiocholanolone | |
| 28 | Cortisone | 4-pregnene-17α,21-diol-3,11,20- |
| | | trione |
| 29 | Tetrahydrocortisone | 5β-pregnene-3α,17,21-triol-11,20- |
| | | dione |
| 30 | α-cortolone | 5-pregnane-3α,17,20α,21-tetrol-11 |
| | | one |
| 31 | β-cortolone | 5β-pregnane-3α,17,20β,21-tetrol- |
| | | 11-one |
| 32 | 11-oxoetiocholanolone | 5β-androstan-3α-ol-11,17-dione |

GMLVQ Analysis of the Urinary Steroid Metabolome can Distinguish Early from Advanced Fibrosis.

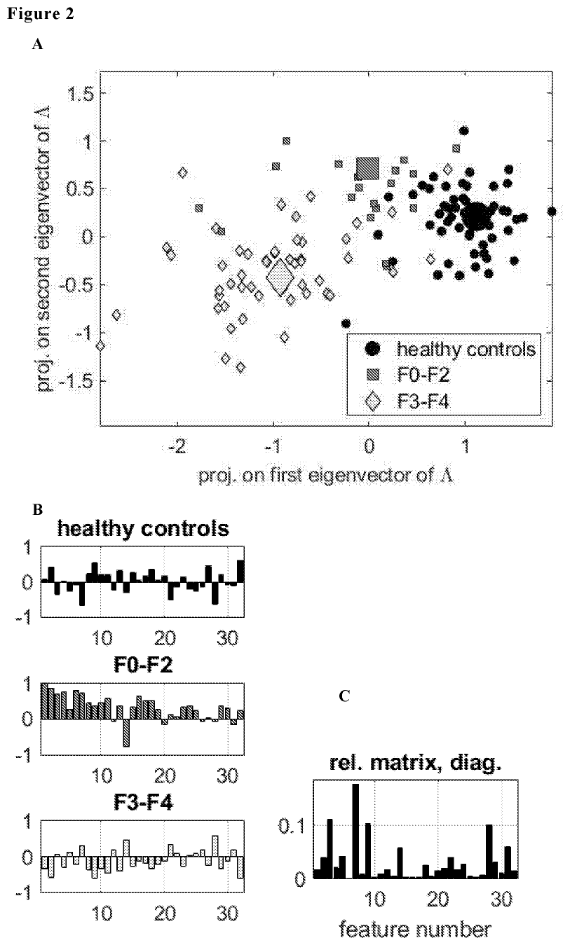

Analysis of data using individual steroid metabolites and ratios demonstrated significant overlap across all groups and therefore there was limited potential to be able to correctly determine NAFLD disease stage. A global approach was therefore adopted, which used GMLVQ to analyse all 32 urinary steroids and metabolites (FIG. 2A) based on the generation of prototype steroid profiles (FIG. 2B) and a relevance matrix which indicates the importance of individual steroids to the GMLVQ classifier (FIG. 2C).

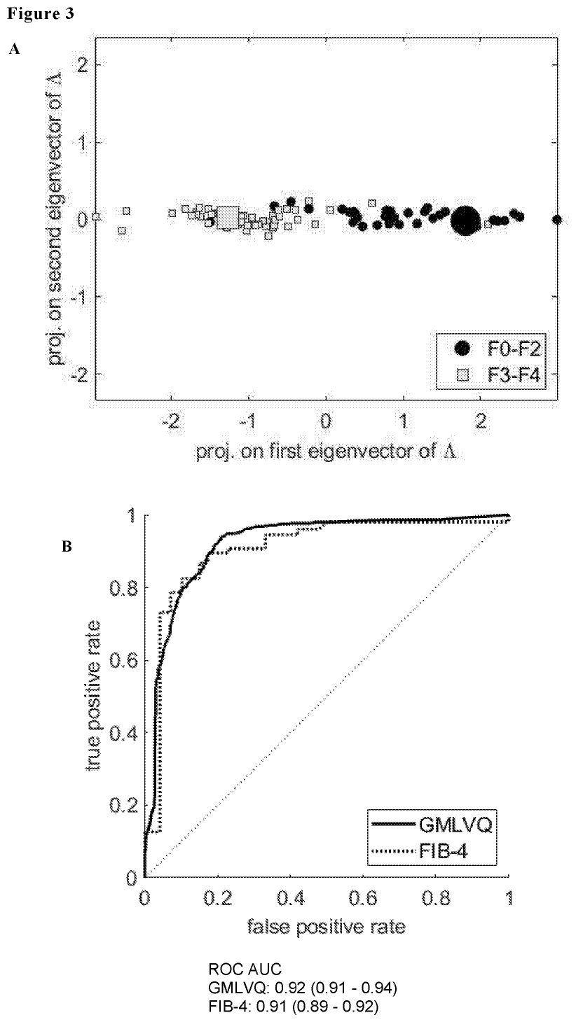

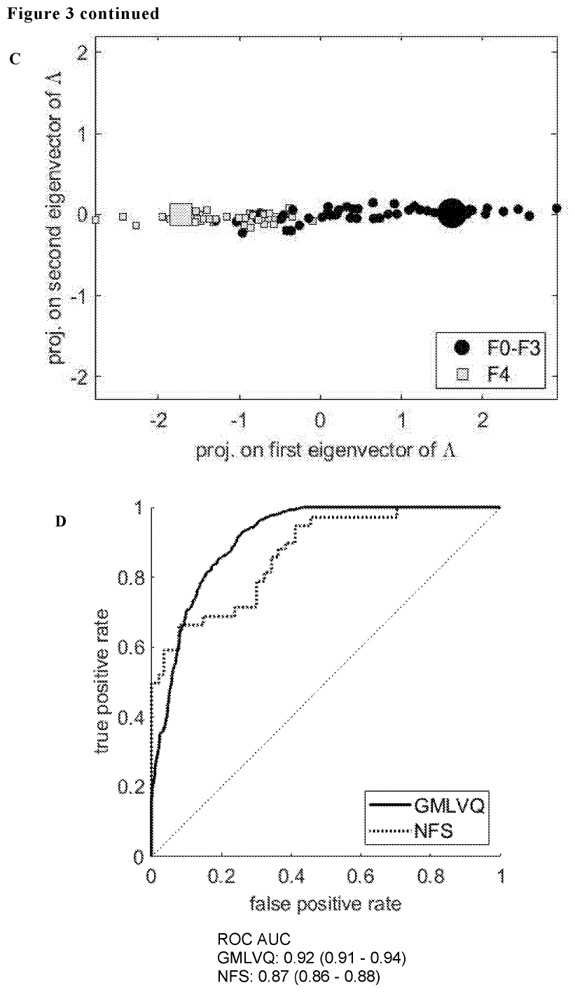

GMLVQ performance was further enhanced by the inclusion of both age and body mass index (BMI) into the model (GMLVQ*) (Table 4). In order to address the binary problem of identifying those individuals with established NAFLD who have either early (F0-2) vs. advanced (F3-4) fibrosis, 2D representative plots were produced as shown in FIG. 3A which demonstrated good separation. Corresponding area under the curve (AUC) analysis of the receiver operating characteristics (ROC) curves suggested that urinary steroid GMLVQ and GMLVQ* performed as well as the established non-invasive serum marker algorithm, Fib-4 (FIG. 3B) (Table 4).

TABLE 4
Comparison of GMLVQ analysis of urinary steroid metabolites vs.
serum assessments using Fib4 and NAFLD fibrosis scores
(Analysis of samples corrected for urinary creatinine).

| | AUC ROC (95% confidence intervals) | | | | | |
|---|---|---|---|---|---|---|
| | | | | | GMLVQ- | GMLVQ- |
| | | | | GMLVQ* | 10 | 10* |
| | | | | (32 | (top 10 | (top 10 |
| Clinical | NAFLD | | GMLVQ | steroids, | steroid | steroid |
| compar- | Fibrosis | FIB- | (32 | age, | metabo- | metabolites, |
| ison | score | 4 | steroids) | BMI) | lites) | age, BMI) |
| F0-F2 | 0.87 | 0.91 | 0.89 | 0.92 | 0.87 (0.85- | 0.92 (0.91- |
| vs. | (0.86- | (0.89 | (0.87- | (0.91- | 0.88) | 0.94) |
| F3-F4 | 0.88) | 0.92) | 0.90) | 0.94) | | |
| F0-F3 | 0.87 | 0.84 | 0.87 | 0.92 | 0.85 (0.83- | 0.90 (0.89- |
| vs. F4 | (0.86- | (0.83 | (0.85- | (0.91- | 0.87) | 0.92) |
| | 0.88) | 0.85) | 0.89) | 0.94) | | |

| | | | | |
|---|---|---|---|---|
| Controls | 0.93 | 0.94 | 0.92 (0.91- | 0.94 (0.93- |
| vs. | (0.92- | (0.92- | 0.93) | 0.96) |
| F0-F4 | 0.94) | 0.95) | | |
| Controls | 0.99 | 0.98 | 0.99 (0.98- | 0.98 (0.98- |
| vs. | (0.98- | (0.97- | 0.99) | 0.99) |
| F3-F4 | 0.99) | 0.98) | | |
| Controls | 1.00 | 1.00 | 1.00 (1.00- | 1.00 (0.99- |
| vs. F4 | (1.00- | (1.00- | 1.00) | 1.00) |
| | 1.00) | 1.00) | | |

Patients with liver cirrhosis are at a higher risk of developing hepatocellular carcinoma and hepatic decompensation and therefore require active monitoring and surveillance. GMLVQ and GMLVQ* were able to identify those patients with NAFLD cirrhosis (F0-3 vs. F4) and out-performed non-invasive serological assessments including NAFLD fibrosis score and Fib-4 (FIG. 3C and D, Table 4).

GMLVQ Analysis of the Urinary Steroid Metabolome has Excellent Potential to Identify Patients with Advanced NAFLD in the General Population.

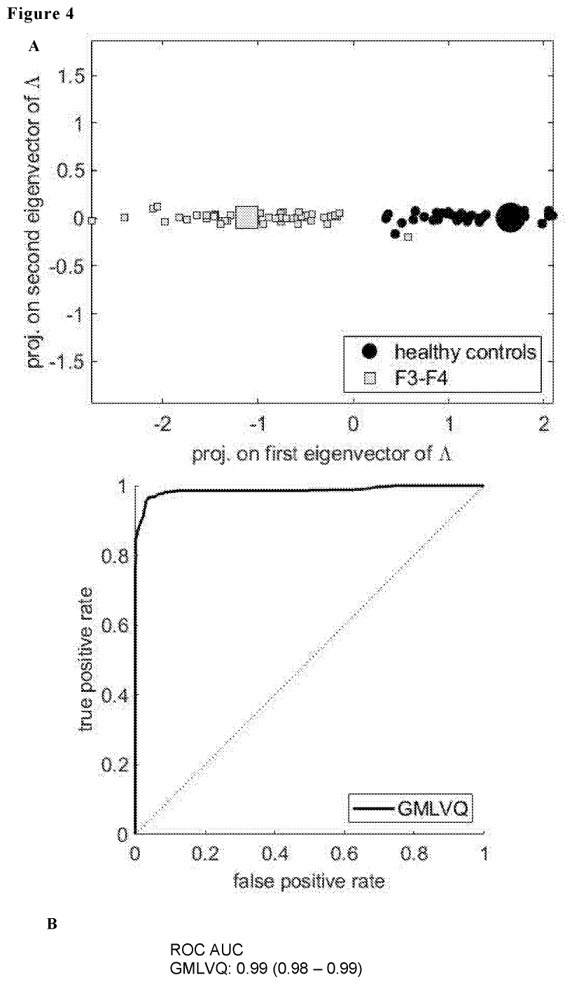

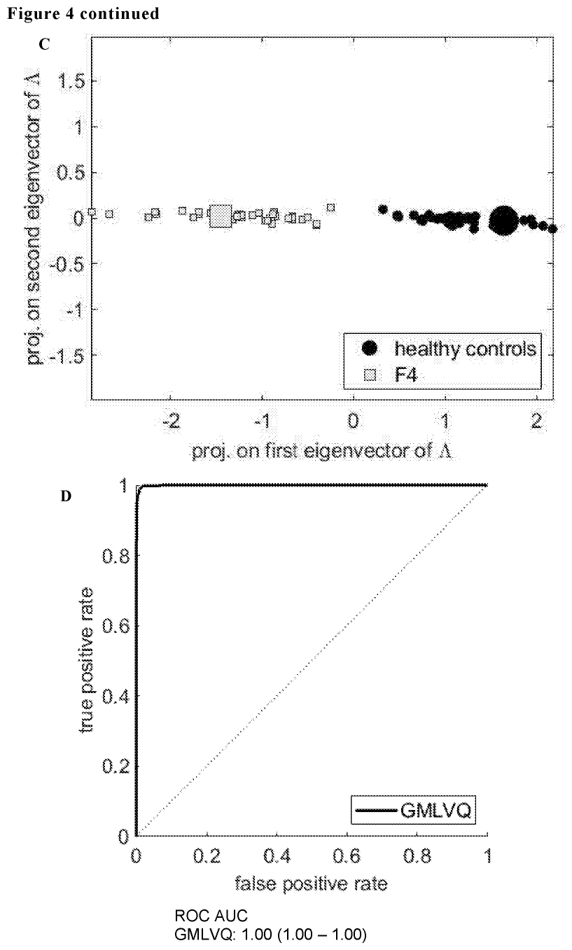

Studies have suggested a high prevalence of undiagnosed advanced NALFD in the general population Armstrong M J et al., J. Hepatol; 56(1): 234-40 and Caballeria L et al., Clin Gastroenterol Hepatol 2018; 16(7): 1138-45 e5), and whilst screening is not currently advocated, identification of advanced fibrosis and cirrhosis would significantly alter patient management. Both GMLVQ and GMLVQ* demonstrated excellent separation and diagnostic ability in identifying patients with advanced NAFLD when compared with healthy controls (FIG. 4A and B). When used to identify those patients with NASH cirrhosis, there was perfect separation and AUC ROC=1.0 (1.00-1.00, 95% confidence intervals) (FIG. 4C and D) (Table 4).

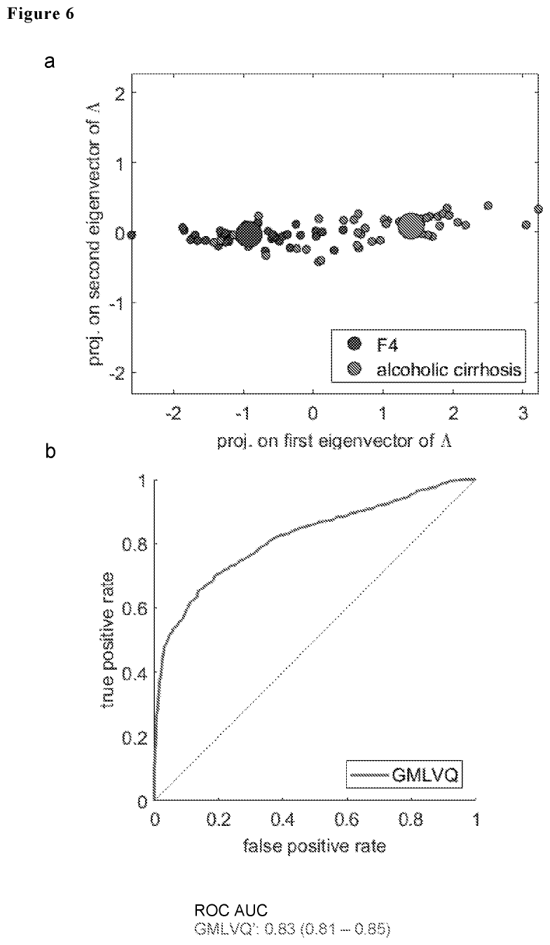

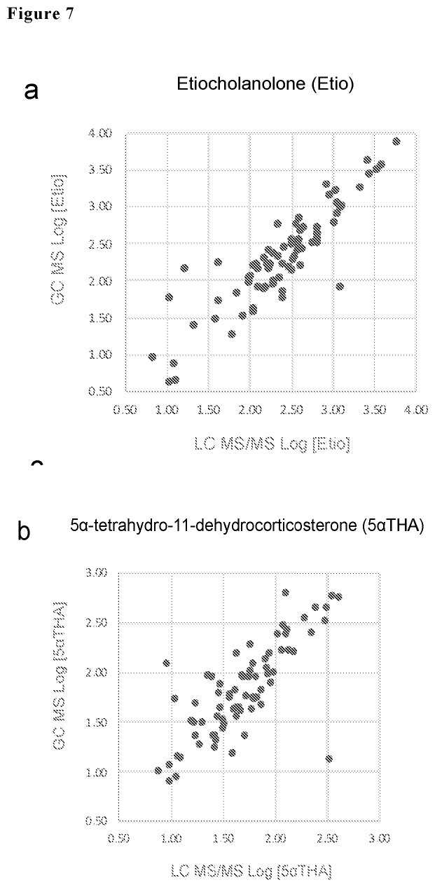

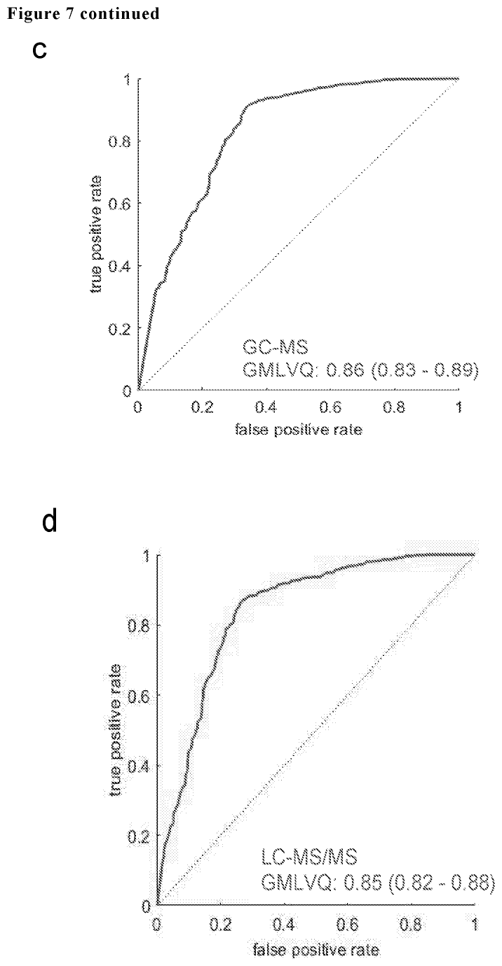

In order to determine if GMLVQ* of urinary steroid metabolite data could identify the underlying aetiology of cirrhosis, a further analysis comparing samples from patients with NAFLD cirrhosis to those from patients with alcohol-related cirrhosis was performed (Table 6). GMLVQ* demonstrated good separation and diagnostic ability to differentiate the underlying aetiology of cirrhosis (AUC ROC=0.83 [0.81-0.85, 95% confidence intervals], FIG. 6).

Additional analyses were also performed separating data by gender as well as comparing urinary steroid metabolites uncorrected for urinary creatinine. No impact of gender was found (data not shown) and AUC ROC analysis was similar using data from samples where uncorrected steroid metabolite levels were expressed as μg steroid/1000 mL urine (Table 1).

GMVLQ can be Refined to Include Only 10 Urinary Steroid Metabolites without Significant Loss in Diagnostic Performance

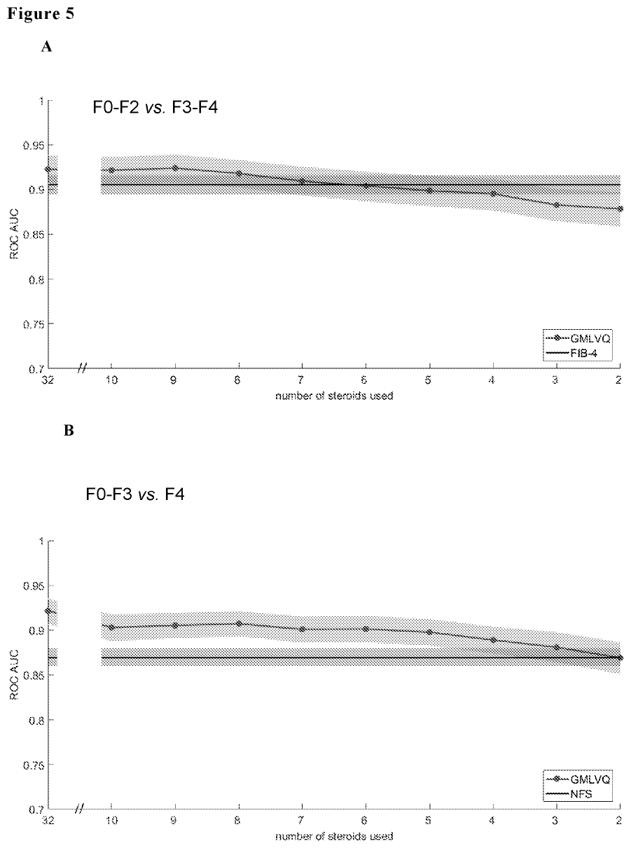

A further GMLVQ analysis was performed with sequential removal of the least discriminatory steroid metabolites. GMLVQ analysis was then compared against the best performing non-invasive serum markers (Fib-4 for F0-2 vs. F3-4 and NAFLD fibrosis score for F0-3 vs. F4). Refining the model from 32 metabolites to 10 (GMLVQ-10) did not result in any loss of diagnostic performance and GMLVQ analysis incorporating age and BMI using 10 steroid metabolites (GMLVQ-10*) still out-performed FIB-4 (F0-2 vs. F3-4) and NAFLD fibrosis score (F0-3 vs. F4) (FIGS. 5A and B respectively) (Table 4). In addition, the analysis of 10 most discriminatory steroids was still able to distinguish NAFLD cirrhosis from alcohol-related cirrhosis (GMLVQ-10*; AUC ROC=0.82 [0.81-0.84, 95% confidence intervals]). The 10 most discriminatory steroids that had the most impact in distinguishing each of the clinical comparisons (F0-2 vs. F3-4; F0-3 vs. F4; Healthy control vs. F3-4; Healthy control vs. F4) are shown in Table 5.

TABLE 5
GMLVQ analysis identifies the 10 most discriminatory steroid metabolites
for distinguishing clinically relevant stages of NAFLD. Steroids
highlighted in bold are common to all clinical comparisons

| Discrimi- | NAFLD stage comparison | | | |
|---|---|---|---|---|
| natory | | | Healthy control | Healthy control |
| ranking | F0-2 vs. F3-4 | F0-3 vs. F4 | vs. F3-4 | vs. F4 |
| 1 | Etiocholanolone | Etiocholanolone | 5α-tetrahydro-11- | 5α-tetrahydro-11- |
| | | | dehydrocorticosterone | dehydrocorticosterone |
| 2 | Dehydro- | Tetrahydro- | 11- | 11- |
| | epiandrosterone | corticosterone | oxoetiocholanolone | oxoetiocholanolone |
| 3 | 5α-tetrahydro- | 5α-tetrahydro- | Etiocholanolone | Etiocholanolone |
| | 11-dehydro- | 11-dehydro- | | |
| | corticosterone | corticosterone | | |
| 4 | Androstendione | Tetrahydro-11 | Cortisone | Cortisone |
| | | deoxycorticosterone | | |
| 5 | 5α- | Dehydro- | Pregnenediol | Tetrahydro-11 |
| | tetrahydro- | epiandrosterone | | deoxycorticosterone |
| | corticosterone | | | |
| 6 | Pregnenetriol | Androstendione | Pregnanetriol | Pregnenediol |
| 7 | Tetrahydro-11 | Tetrahydrocortisone | Tetrahydro-11 | Pregnanetriol |
| | deoxy- | | deoxycorticosterone | |
| | corticosterone | | | |
| 8 | Tetrahydro- | Tetrahydrocortisol | 11β-hydroxy- | Tetrahydro- |
| | aldosterone | | etiocholanolone | corticosterone |
| 9 | Cortisone | Pregnenetriol | Pregnanediol | Pregnanediol |
| 10 | 11- | 5α- | 5α- | 5α- |
| | oxoetiocholanolone | tetrahydro- | tetrahydro- | tetrahydro- |
| | | corticosterone | corticosterone | corticosterone |

TABLE 6
Demographic details of 108 subjects with cirrhosis (F4):
60 with NAFLD cirrhosis and 48 with cirrhosis due to
excess alcohol consumption. Data are expressed are mean ±
standard deviation (unless otherwise stated) (*p < 0.05).

| | NAFLD | Alcohol | p- |
|---|---|---|---|
| | cirrhosis | cirrhosis | value |
| N (m/f) (males, %) | 60 (26/34)(43) | 48 (33/15) (69) | 0.29 |
| Age, years | 65 ± 9 | 58 ± 11 | <0.01 |
| BMI, kg/m² | 33.2 ± 5.9 | 28.2 ± 5.9 | <0.01 |
| Proportion with | 70 | 26 | <0.01 |
| Type 2 Diabetes, % | | | |
| Platelets, 10⁹/L | 173 ± 67 | 146 ± 43 | 0.21 |
| ALT, IU/L | 39 ± 19 | 33 ± 25 | 0.08 |
| AST, IU/L | 41 ± 17 | 52 ± 56 | 0.32 |
| Fib-4 Score | 2.9 ± 1.8 | 3.2 ± 1.6 | 0.32 |
| NAFLD Fibrosis | 0.90 ± 1.6 | 0.70 ± 1.39 | 0.7 |
| Score | | | |

### Summary

Urinary steroid metabolites were analysed using GC/MS in 121 patients with biopsy-proven NAFLD, 106 healthy control subjects and 48 with alcohol-related cirrhosis. Specific pathway analysis revealed differences in the capacity of the liver to both regenerate, and inactivate steroid hormones in those patients with the most advanced stages of NAFLD, including cirrhosis. Machine learning-based analysis using generalised matrix learning vector quantisation (GMLVQ) achieved excellent separation of early from advanced fibrosis (AUC ROC: 0.92 [0.91-0.94]). Furthermore, there was near perfect separation of healthy controls from patients with both advanced fibrotic NAFLD (AUC ROC=0.99 [0.98-0.99]) as well as from those with NAFLD cirrhosis (AUC ROC=1.0 [1.0-1.0]).

Unbiased GMLVQ analysis of the urinary steroid metabolome appears to offer excellent potential as a non-invasive biomarker to stage NAFLD severity. A urinary biomarker that is both sensitive and specific is likely to have clinical utility both in secondary care as well as in the broader general population and could significantly decrease the need for liver biopsy.

### DISCUSSION

The relationship between NAFLD disease stage and clinical outcome is now well established (Dulai P S et al., Hepatology, 2017; 65(5): 1557-65 and Ekstedt M et al., Hepatology 2014). If appropriate management strategies are to be implemented, investigative and disease monitoring tools that do not carry the associated risks and limitations of liver biopsy are needed. There is a therefore a pressing need for the development of accurate non-invasive markers of stage of liver disease, fueled, at least in part, by the poor performance of simple routine liver biochemistry. There are many serological tests, algorithms and imaging modalities that perform reasonably well in their ability to identify disease severity and stage. Imaging modalities including magnetic resonance spectroscopy (MRS) and imaging (MRI) provide accurate assessment of hepatic triglyceride content (Bannas et al., Hepatology 2015; 62(5): 1444-55). Identifying inflammation within the liver is more challenging and whilst there is some potential from novel imaging platforms and serological tests (for example the measurement of cytokeratin-18 fragments or cathepsin D (Walenbergh et al., Am. J. Gastroenterol. 2015; 110(3): 462-70), AUC ROC analysis is less impressive than non-invasive biomarkers to stage fibrosis.

The number of potential tests that can be used to assess the risk of advanced fibrosis is large. Data from more than 20 different tests, algorithms or imaging platforms have been published and the large number of tests perhaps reflects the need for improved performance. The range of ROC AUC values is broad for many of these tests that are currently used in clinical practice, and the majority of studies suggest values between 0.8 and 0.9. The use of a urinary test as defined herein is novel.

Urinary steroid metabolome analysis using GMLVQ has been used to help differentiate benign from malignant adrenal tumours, but its use in the context of NAFLD is entirely novel. Data from this study (AUC ROC>0.9) shows that GMLVQ analysis of urinary steroids and metabolites thereof can accurately identify subjects with advanced fibrosis. Furthermore, it performs as an almost perfect test in the identification of patients with advanced fibrosis and cirrhosis when compared against a healthy control population. This allows the identification of patients within the general population that have the most advanced liver disease that are at high risk of cardiovascular and hepatic co-morbidities and complications. Estimates suggest that prevalence of compensated cirrhosis is likely to rise in the general population by more than 150% in some countries over the next 10-15 years and therefore identification of these patients is of huge clinical significance.

The principle underpinning the above observations may be transferred to a high-throughput liquid chromatography tandem mass spectrometry approach (Marcos et al., Anal Chim Acta 2014; 812: 92-104) which offers significant savings both in terms of cost and time and would thus increase appeal for future routine clinical use. In conclusion, herein described is an improved, non-invasive approach to accurately determine the presence and stage of NAFLD using the measurement of urinary steroid metabolites.

1. A method of diagnosing non-alcoholic fatty liver disease (NAFLD) in a subject, and/or determining the stage of NAFLD in a subject diagnosed with NAFLD, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof; and
iv. using the results from (iii) to diagnose or determine the stage of non-alcoholic fatty liver disease (NAFLD) in the subject.

2. A method of identifying a subject having an increased risk of developing liver cancer, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof;
iv. using the results from (iii) to diagnose or determine the stage of NAFLD in the subject;
wherein the patient is identified as having an increased risk of liver cancer when the stage of NAFLD is determined to be F3-F4 or F4.

3. A method of treating a subject with NAFLD having advanced fibrosis or cirrhosis, wherein the method comprises:
i. providing a urine sample obtained from the subject;
ii. determining the level of at least one steroid hormone or metabolite thereof in the sample;
iii. comparing the amount of the at least one steroid hormone or metabolite thereof detected in the sample with a reference level of the hormone or the metabolite thereof; and
iv. administering anti-NAFLD therapy to the subject if the level of the hormone or the metabolite thereof is diagnostic of cirrhosis, or the stage of NAFLD is determined as advanced fibrosis or cirrhosis.

4. The method of claim 1, wherein step ii. comprises the steps of: extracting free and conjugated steroid hormones or metabolites thereof from the urine sample and quantifying the steroid hormones or metabolites thereof in the extraction.

5. The method of claim 1, wherein step ii. comprises the steps of
a. extracting free and conjugated steroid hormones or metabolites thereof, for example by solid phase extraction, from the urine sample;
b. hydrolysing the extracted conjugated steroid hormones or metabolites thereof, for example by enzymatic hydrolysis;
c. re-extracting the hydrolysed conjugates of steroid hormones or metabolites thereof, for example using solid phase extraction;
d. performing chemical derivatization on the free and hydrolysed conjugates of steroid hormones or metabolites thereof, to form ethers;
e. performing liquid-liquid extraction; and
f. quantifying the steroid hormones or metabolites thereof in the extraction, for example by using GC/MS (Gas Chromatography/Mass Spectrometry).

6. The method of claim 1, wherein the subject is monitored after the method is undertaken, to assess the efficacy of any treatment suggested or administered and/or or disease progression.

7. The method of claim 6, wherein the monitoring comprises ultrasound scans.

8. The method of claim 1, wherein the level of at least 2 steroid hormones or metabolites thereof in a urine sample are determined.

9. The method of claim 1, wherein the at least one steroid hormone or metabolite thereof is selected from the group comprising androstendione, etiocholanolone, 11β-hydroxyandrosterone, dehydroepiandrosterone, 16α-hydroxy-dehydroepiandrosterone, pregnenetriol, pregnenediol, tetrahydro-11-dehydrocorticosterone, 5α-tetrahydro-11-dehydrocorticosterone, tetrahydrocorticosterone, 5α-tetrahydrocorticosterone, 18-hydroxytetrahydro-11-dehydrocorticosterone, tetrahydro-11 deoxycorticosterone, tetrahydroaldosterone, pregnanediol, 3α,5α-17-hydroxypregnanolone, 17-hydroxypregnanolone, pregnanetriol, pregnanetriolone, tetrahydro-11-deoxycortisol, cortisol, 6β-hydroxy-cortisol, tetrahydrocortisol, 5α-tetrahydrocortisol, α-cortol, β-cortol, 11β-hydroxyetiocholanolone, cortisone, tetrahydrocortisone, α-cortolone, β-cortolone and 11-oxoetiocholanolone.

10. The method of claim 9, wherein the at least one steroid hormone or metabolite thereof is one, two, three, or all of 5α-tetrahydro-11-dehydrocorticosterone, etiocholanolone, pregnanetriol and 5α-tetrahydrocorticosterone.

11. The method of claim 9, wherein the level of 7 steroid hormones or metabolites thereof in a urine sample are determined.

12. The method of claim 1, wherein the urine sample size is between about 1 mL and about 3 mL.

13. The method of claim 1, wherein the sample is fresh, stored for up to 1 hour at −80° C. before undertaking the method.

14. The method of claim 1, wherein the method is carried out in vitro.

15. The method of claim 1, wherein GMLVQ analysis is carried out on the level of steroid hormones or metabolites thereof determined in the urine sample.

16. The method of claim 1, wherein the subject is given a prognosis based on the stage of NAFLD determined.

17. The method of claim 7, wherein the ultrasound seams are taken at about six month intervals after the method is undertaken.
